(19) United States
(12) Patent Application Publication
(10) Pub. No.: US 2017/0348269 A1
(43) Pub. Date: Dec. 7, 2017

(54) TREATMENT OF UREA CYCLE DISORDERS IN NEONATES AND INFANTS

(71) Applicant: Horizon Therapeutics, LLC, Lake Forest, IL (US)

(72) Inventors: Bruce SCHARSCHMIDT, San Francisco, CA (US); Masoud MOKHTARANI, Walnut Creek, CA (US)

(21) Appl. No.: 15/684,876

(22) Filed: Aug. 23, 2017

Related U.S. Application Data
(63) Continuation-in-part of application No. PCT/US2017/018958, filed on Feb. 22, 2017.
(60) Provisional application No. 62/298,222, filed on Feb. 22, 2016.

(51) Int. Cl. A61K 31/216; G01N 33/50; A61K 9/00; G01N 33/84

(52) U.S. Cl. CPC A61K 31/216; G01N 33/84; G01N 33/50; A61K 9/0053; G01N 2800/52; G01N 2800/708

(57) ABSTRACT

Nitrogen scavenging drugs such as glycerol phenylbutyrate can be administered safely to infants and toddlers with urea cycle disorders by adjusting the dosage based on one or more biomarkers selected from the group consisting of urinary-PAGN and plasma PAA:PAGN ratio.

### CROSS-REFERENCE TO RELATED APPLICATIONS

This application is a Continuation-In-Part of PCT International Application No. PCT/US17/18958, filed Feb. 22, 2017, which claims the priority benefit of U.S. provisional application 62/298,222, filed Feb. 22, 2016, the disclosures of which are incorporated herein by reference in their entirety.

### BACKGROUND

Urea cycle disorders (UCD) are inborn errors of metabolism caused by a deficiency in one of six enzymes or two mitochondrial transport proteins involved in the production of urea, resulting in accumulation of toxic levels of ammonia in the blood (hyperammonemia). UCD subtypes include those caused by an X-linked mutation and corresponding deficiency in ornithine transcarbamylase (OTC) and those caused by autosomal recessive mutations with corresponding deficiencies in argininosuccinate synthetase (ASS), carbamyl phosphate synthetase (CPS), argininosuccinate lyase (ASL), arginase (ARG), N-acetylglutamate synthetase (NAGS), ornithine translocase (HHH), and aspartate glutamate transporter (CITRIN). These are rare diseases, with an overall estimated incidence in the United States of approximately 1 in every 35,000 live births. UCD is suspected when a subject experiences a hyperammonemic event with an ammonia level >100 μmol/L accompanied by signs and symptoms compatible with hyperammonemia in the absence of other obvious causes and generally confirmed by genetic testing.

The severity and timing of UCD presentation vary according to the severity of the deficiency, which may range from minor to extreme depending on the specific enzyme or transporter deficiency, and the specific mutation in the relevant gene. UCD patients may present in the early neonatal period with a catastrophic illness, or at any point in childhood, or even adulthood, after a precipitating event such as infection, trauma, surgery, pregnancy/delivery, or change in diet. Acute hyperammonemic episodes at any age carry the risk of encephalopathy and resulting neurologic damage, sometimes fatal, but even chronic, sub-critical hyperammonemia can result in impaired cognition. UCDs are therefore associated with a significant incidence of neurological abnormalities and intellectual and developmental disabilities over all ages. UCD patients with neonatal-onset disease are especially likely to suffer cognitive impairment and death compared with patients who present later in life.

Management of acute hyperammonemic crises may require hemodialysis and/or intravenous (IV) administration of sodium phenylacetate (NaPAA) and sodium benzoate (NaBz) (the admixture is marketed in the U.S. as AMMONUL®). Orthotopic liver transplantation may also be considered for patients with severe disease that manifests itself in the neonatal period. Long-term UCD management is directed toward prevention of hyperammonemia and includes restriction of dietary protein; arginine and citrulline supplementation, which can enhance waste nitrogen excretion for certain UCDs; and oral, ammonia-scavenging drug therapy that provides an alternate path for waste nitrogen removal (RAVICTI® (glycerol phenylbutyrate, GPB) Oral Liquid or sodium phenylbutyrate (NaPBA; marketed in the U.S. as BUPHENYL® and in the European Union (EU) as AMMONAPS®)).

RAVICTI®, formerly HPN-100, a prodrug of PBA and a pre-prodrug of the active compound phenylacetate (PAA), has been approved in the U.S. for use as a nitrogen-binding agent for chronic management of adults and patients 2 months of age and older with UCDs who cannot be managed by dietary protein restriction and/or amino acid supplementation alone. RAVICTI® is glycerol phenylbutyrate, a triglyceride containing 3 molecules of PBA linked to a glycerol backbone, the chemical name of which is benzenebutanoic acid, 1′,1″-(1,2,3-propanetriyl) ester.

Glycerol phenylbutyrate is used with dietary protein restriction and, in some cases, dietary supplements (e.g., essential amino acids, arginine, citrulline, protein-free calorie supplements). RAVICTI® is not indicated for the treatment of acute hyperammonemia in patients with UCDs, and the safety and efficacy of RAVICTI® for the treatment of NAGS deficiency has not been established. The RAVICTI® Package Insert states the drug is contraindicated in patients less than 2 months of age, stating that children less than 2 months of age may have immature pancreatic exocrine function, which could impair hydrolysis of RAVICTI®, leading to impaired absorption of phenylbutyrate and hyperammonemia; and in patients with known hypersensitivity to phenylbutyrate (signs include wheezing, dyspnea, coughing, hypotension, flushing, nausea, and rash). Pancreatic lipases may be necessary for intestinal hydrolysis of RAVICTI®, allowing release of phenylbutyrate and subsequent formation of PAA, the active moiety. It is not known whether pancreatic and extrapancreatic lipases are sufficient for hydrolysis of RAVICTI®.

While the U.S. approval of RAVICTI® was based on its evaluation in 6 clinical trials involving over 100 adult and pediatric UCD patients aged 2 months old and above, only 7 patients aged 2 months to 2 years were enrolled in these studies. Among the 4 patients aged 2 months to 2 years who participated in an open label, fixed-sequence switch-over comparison of RAVICTI® to NaPBA, mean ammonia exposure assessed at 24-hour area under the curve was non-inferior on RAVICTI®. PAA exposure, also assessed as 24-hour area under the curve, was very similar. However, the number of patients in this age group studied at the time of RAVICTI's approval was small and considerable patient-to-patient variability was observed.

Children less than 2 months of age may have immature pancreatic exocrine function, which could impair hydrolysis of glycerol phenylbutyrate leading to impaired absorption of PBA and potentially hyperammonemia. While the limited data available suggest that pancreatic enzymes present in newborns include pancreatic lipase-related protein and bile salt-stimulated lipase (which digest triglycerides present in human breast milk), both of which hydrolyze glycerol phenylbutyrate in vitro, it is not known whether pancreatic function in newborns is sufficiently mature to digest glycerol phenylbutyrate. In addition, the metabolism of PAA is known to vary with body size, and given that body size changes dramatically during the first two years of life, there has been significant uncertainty how glycerol phenylbutyrate or NaPBA could be used in these patient populations from birth to two years of age.

There is a significant, unmet need for a nitrogen scavenging drug that can be used in UCD patients less than 2 years of age. Further, for the purposes of dose monitoring, frequent blood draws are difficult in neonates and children under two years of age and a non-invasive measure is needed. The present disclosure meets these needs.

### SUMMARY

Provided is the use of glycerol phenylbutyrate as a nitrogen-binding agent for chronic management of pediatric patients with a UCD under 2 years in age.

Also provided is a method of treating a UCD in a patient under 2 years of age comprising administering glycerol phenylbutyrate to said patient at an initial daily dose which is administered at an initial frequency of administration; and if after said administration, said patient exhibits a urinary phenylacetylglutamine (PAGN) ≦9000 μg/mL and a ratio of plasma PAA to plasma PAGN of ≦2.5, then administering said glycerol phenylbutyrate to said patient at an increased daily dose while maintaining said initial frequency of administration. In some embodiments, the patient exhibits elevated blood ammonia levels. In some embodiments, the compliance and effectiveness of drug delivery by the patients' parent(s), guardian(s), or health care provider(s) is also assessed. In some embodiments, urinary PAGN concentration is measured using a filter paper-type test. In some embodiments, urinary PAGN concentration is measured from urine collected directly from a diaper or via the blood-spot technology commonly used for The Newborn Screen (NBS).

Also provided is a method of treating a UCD in a patient under 2 years of age comprising administering glycerol phenylbutyrate to said patient at an initial daily dose which is administered at an initial frequency of administration; and if after said administration, said patient exhibits a urinary PAGN≧9000 μg/mL and a ratio of plasma PAA to plasma PAGN of >2.5, then administering said glycerol phenylbutyrate to said patient at an increased frequency of administration while maintaining said daily dose. In some embodiments, the patient exhibits elevated blood ammonia levels. In some embodiments, the compliance and effectiveness of drug delivery by the patients' parent(s), guardian(s), or health care provider(s) is also assessed. In some embodiments, urinary PAGN concentration is measured using a filter paper-type test. In some embodiments, urinary PAGN concentration is measured from urine collected directly from a diaper or via the blood-spot technology commonly used for the NBS.

Also provided is a method of treating a UCD in a patient under 2 years of age comprising administering glycerol phenylbutyrate to said patient at an initial daily dose which is administered at an initial frequency of administration; and if after said administration, said patient exhibits a ratio of plasma PAA to plasma PAGN of >2.5 or a level of plasma PAA of >500 μg/mL, then administering said glycerol phenylbutyrate to said patient at a reduced daily dose while maintaining said initial frequency of administration. In some embodiments, the patient exhibits neurological symptoms such as vomiting, headache, lethargy, and/or somnolence. In some embodiments, the patient exhibits normal blood ammonia levels. In some embodiments, the compliance and effectiveness of drug delivery by the patients' parent(s), guardian(s), or health care provider(s) is also assessed. In some embodiments, urinary PAGN concentration is measured using a filter paper-type test. In some embodiments, urinary PAGN concentration is measured from urine collected directly from a diaper or via the blood-spot technology commonly used for the NBS.

Also provided is a method of treating a UCD in a patient under 2 years of age comprising administering glycerol phenylbutyrate to said patient at an initial daily dose which is administered at an initial frequency of administration; and if after said administration, said patient exhibits a ratio of plasma PAA to plasma PAGN of >2.5 or a level of plasma PAA of >500 μg/mL, then administering said glycerol phenylbutyrate to said patient at an increased frequency of administration while maintaining said daily dose. In some embodiments, the patient exhibits neurological symptoms. In some embodiments, the patient exhibits normal blood ammonia levels. In some embodiments, the compliance and effectiveness of drug delivery by the patients' parent(s), guardian(s), or health care provider(s) is also assessed. In some embodiments, urinary PAGN concentration is measured using a filter paper-type test. In some embodiments, urinary PAGN concentration is measured from urine collected directly from a diaper or via the blood-spot technology commonly used for the NBS.

Also provided is a method of treating a UCD in a patient under 2 years of age comprising administering glycerol phenylbutyrate to said patient at an initial daily dose which is administered at an initial frequency of administration; and if after said administration said patient exhibits neurological symptoms, normal ammonia levels, and a ratio of plasma PAA to plasma PAGN of <2.5, then said glycerol phenylbutyrate to said patient should continue to be administered at the same dose and frequency and the patient evaluated for other causes of his or her neurological symptoms. In some embodiments, the patient exhibits neurological symptoms. In some embodiments, the patient exhibits normal blood ammonia levels. In some embodiments, the compliance and effectiveness of drug delivery by the patients' parent(s), guardian(s), or health care provider(s) is also assessed. In some embodiments, urinary PAGN concentration is measured using a filter paper-type test. In some embodiments, urinary PAGN concentration is measured from urine collected directly from a diaper or via the blood-spot technology commonly used for the NBS.

Also provided is a method of treating a UCD in a patient under 2 years of age comprising administering glycerol phenylbutyrate to said patient at an initial daily dose which is administered at an initial frequency of administration; and if after said administration, said patient exhibits elevated ammonia levels, a urinary PAGN<9000 μg/mL, a ratio of plasma PAA to plasma PAGN of ≦2.5, and compliance with initial daily dosing is confirmed, then administering said glycerol phenylbutyrate to said patient at an increased dose while maintaining frequency of administration. In some embodiments, the patient exhibits elevated blood ammonia levels. In some embodiments, the compliance and effectiveness of drug delivery by the patients' parent(s), guardian(s), or health care provider(s) is also assessed. In some embodiments, urinary PAGN concentration is measured using a filter paper-type test. In some embodiments, urinary PAGN concentration is measured from urine collected directly from a diaper or via the blood-spot technology commonly used for the NBS.

Also provided is a method of treating a UCD in a patient under 2 years of age comprising administering glycerol phenylbutyrate to said patient at an initial daily dose which is administered at an initial frequency of administration; and if after said administration, said patient exhibits elevated ammonia levels, a urinary PAGN<9000 μg/mL, and a ratio of plasma PAA to plasma PAGN of >2.5, then administering said glycerol phenylbutyrate to said patient at an increased dose frequency while maintaining the said daily dose. In some embodiments, the patient exhibits elevated blood ammonia levels. In some embodiments, the compliance and effectiveness of drug delivery by the patients' parent(s), guardian(s), or health care provider(s) is also assessed. In some embodiments, urinary PAGN concentration is measured using a filter paper-type test. In some embodiments, urinary PAGN concentration is measured from urine collected directly from a diaper or via the blood-spot technology commonly used for the NBS.

Also provided is a method of administering glycerol phenylbutyrate to a patient in need thereof comprising administering daily to the patient a therapeutically effective amount of the glycerol phenylbutyrate in three or more equally divided doses. In some embodiments, the patient is between 2 months of age to less than 2 years of age.

In some embodiments, the method further comprises restricting the patient's dietary protein. In some embodiments, the method further comprises monitoring the patient's plasma ammonia levels to determine the need for dosage titration of the glycerol phenylbutyrate. In some embodiments, the method further comprises adjusting the glycerol phenylbutyrate dosage to keep the first ammonia of the morning below the upper limit of normal.

In some embodiments, the glycerol phenylbutyrate is administered orally. In some embodiments, the glycerol phenylbutyrate is administered using a nasogastric and/or gastronomy tube. In some embodiments, the volume of glycerol phenylbutyrate administered is less than 1 mL per dose. In some embodiments, the method further comprises monitoring the patient's ammonia level.

In some embodiments, the patient was previously administered phenylbutyrate. In some embodiments, the patient previously had not been administered phenylbutyrate.

In some embodiments, the therapeutically effective amount of the glycerol phenylbutyrate is 4.5 to 11.2 mL/m²/day (5 to 12.4 g/m²/day). In some embodiments, the therapeutically effective amount of the glycerol phenylbutyrate is 4.8 to 11.5 mL/m²/day (5.3 to 12.6 g/m²/day). In some embodiments, the therapeutically effective amount of the glycerol phenylbutyrate is 3.3 to 12.3 mL/m²/day (3.7 to 13.5 g/m²/day). In some embodiments, the therapeutically effective amount of the glycerol phenylbutyrate is 4.5 mL/m²/day (5 g/m²/day). In some embodiments, the therapeutically effective amount of the glycerol phenylbutyrate is 8 mL/m²/day (8.8 g/m²/day). In some embodiments, the therapeutically effective amount of the glycerol phenylbutyrate is 7.5 mL/m²/day (8.2 g/m²/day).

In some embodiments, the method further comprises obtaining measurements of plasma phenylacetate (PAA) concentrations and the ratio of plasma PAA to phenylacetylglutamine (PAGN). In some embodiments, the method further comprises obtaining measurements of urinary phenylacetylglutamine (U-PAGN). In some embodiments, if the U-PAGN excretion is insufficient to cover daily dietary protein intake and/or the fasting ammonia is greater than half the upper limit of normal, the method further comprises increasing the glycerol phenylbutyrate dosage.

In some embodiments, the glycerol phenylbutyrate is administered just prior to breastfeeding or intake of formula or food. In some embodiments, the glycerol phenylbutyrate is administered three to six times daily.

These and other embodiments of the disclosure are described in detail below.

### BRIEF DESCRIPTION OF THE DRAWINGS

FIG. 1 shows mean±SE blood ammonia values for a 24-hour period following 7 days of treatment with NaPBA and HPN-100 in pediatric UCD subjects (N=11).

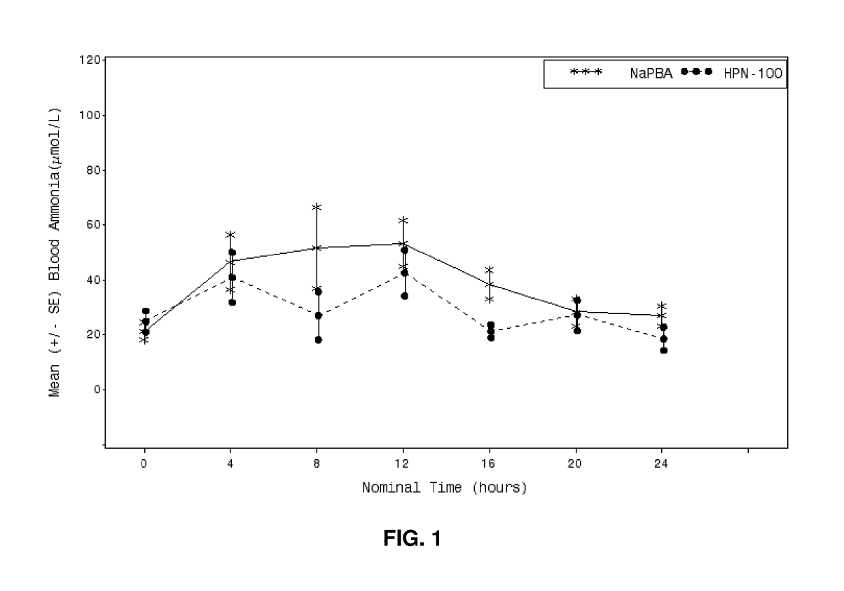

FIG. 2 shows mean blood ammonia over 24-h after treatment with NaPBA or HPN-100 (ITT Population). HPN-100=glycerol phenylbutyrate; ITT=intent-to-treat; NaPBA=sodium phenylbutyrate; SE=standard error; UCD=urea cycle disorder; ULN=upper limit of normal.

FIG. 3 shows a flow chart for dosing based on PAA:PAGN ratio in infants and toddlers presenting with neurological symptoms and normal ammonia levels.

FIG. 4 shows a flow chart for dosing based on U-PAGN ratio and plasma PAA:PAGN ratio in infants and toddlers with elevated ammonia.

FIG. 5 shows a chart with UCD patient information relating to patient presentation and contributors of delayed- and mis-diagnosis.

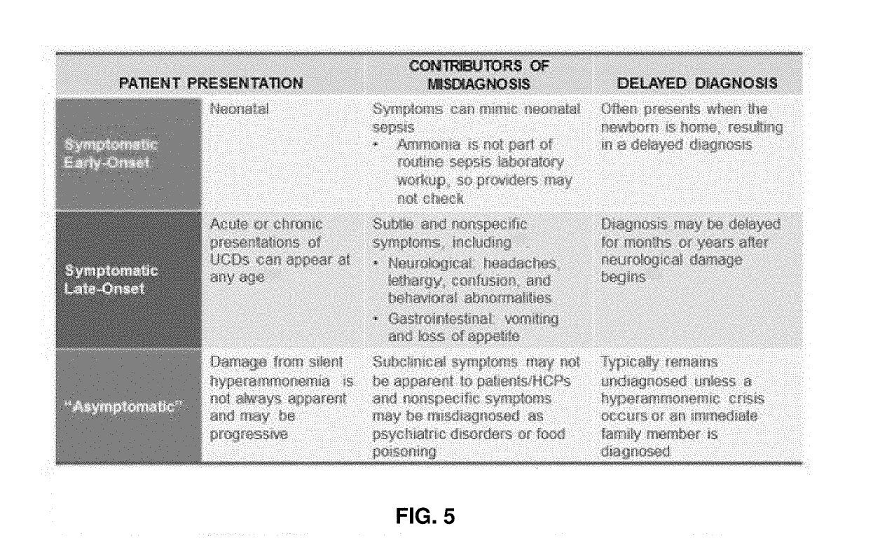

FIG. 6 shows a chart with the spectrum of UCD signs and symptoms beyond hyperammonemic (HA) crises.

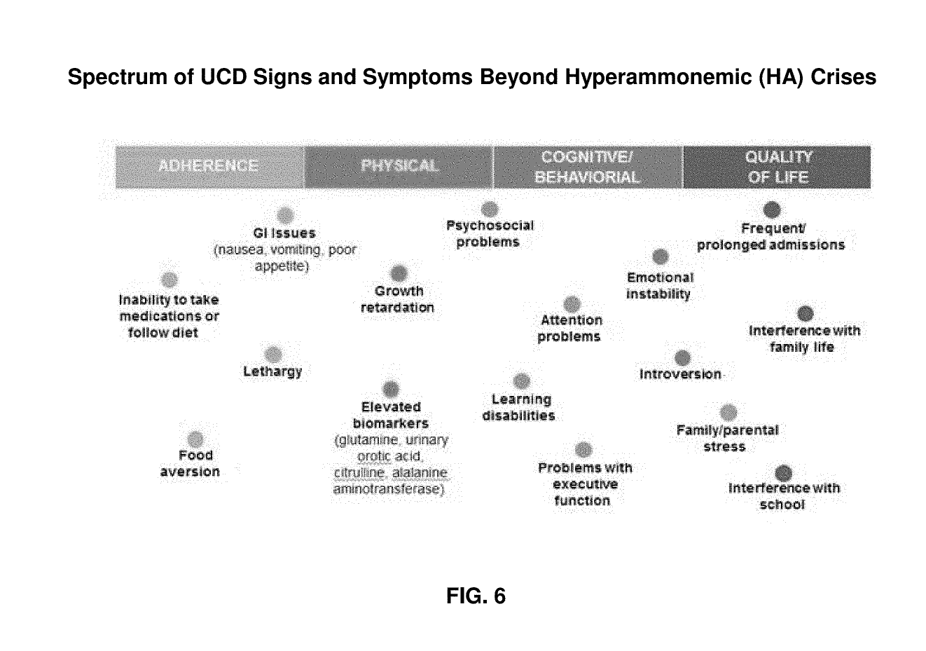

FIG. 7 shows a chart with UCD patient symptom and disease progression information.

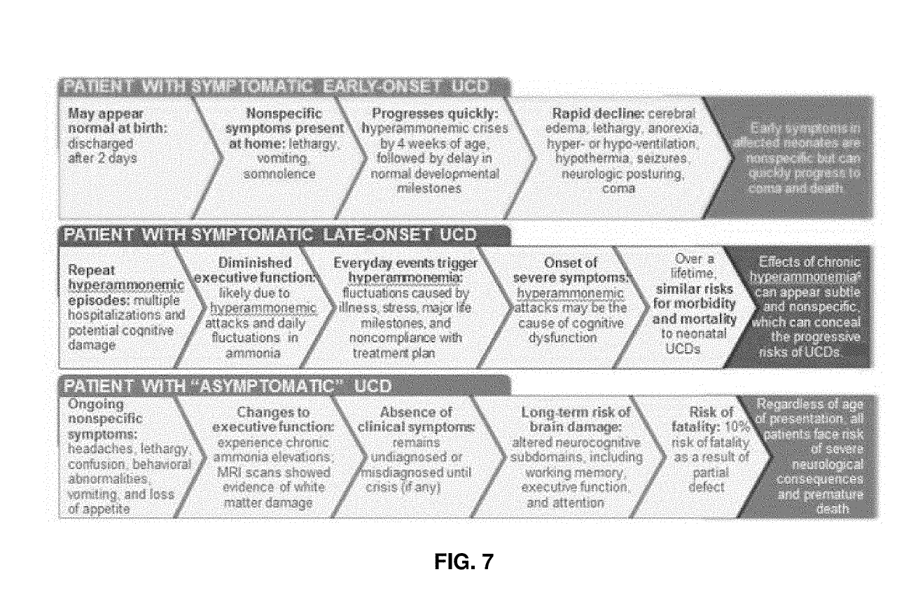

### DETAILED DESCRIPTION

As used in this specification and the appended claims, the singular forms “a,” “an” and “the” include plural referents unless the context clearly dictates otherwise. Thus, for example, “an active agent” refers not only to a single active agent but also to a combination of two or more different active agents, “a dosage form” refers to a combination of dosage forms as well as to a single dosage form, and the like.

Unless defined otherwise, all technical and scientific terms used herein have the meaning commonly understood by one of ordinary skill in the art to which the disclosure pertains. Specific terminology of particular importance to the description of the present disclosure is defined below.

The terms “treating” and “treatment” as used herein refer to reduction in severity and/or frequency of symptoms, elimination of symptoms and/or underlying cause, and improvement or remediation of damage. In certain aspects, the term “treating” and “treatment” as used herein refer to the prevention of the occurrence of symptoms. In other aspects, the term “treating” and “treatment” as used herein refer to the prevention of the underlying cause of symptoms associated with obesity, excess weight, and/or a related condition. The phrase “administering to a patient” refers to the process of introducing a composition or dosage form into the patient via an art-recognized means of introduction.

By the terms “effective amount” and “therapeutically effective amount” of an agent, compound, drug, composition or combination which is nontoxic and effective for producing some desired therapeutic effect upon administration to a subject or patient (e.g., a human subject or patient).

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

As used herein, “subject” or “individual” or “patient” refers to any patient for whom or which therapy is desired, and generally refers to the recipient of the therapy.

With regards to “patients under 2 years of age”, the International Conference on Harmonisation (ICH) categorizes the pediatric population as follows: newborn (birth to 1 month), infants/toddlers (1 month to <24 months), children (2 years to <12 years), and adolescents (12 years to <16 years).

As used herein, the term “normal ammonia levels” refers to a patient's blood plasma ammonia concentration less than 35 μmol/L. As used herein, the term “elevated ammonia levels” refers to refers to a patient's blood plasma ammonia concentration equal to or greater than 35 μmon. In some embodiments, the ULN is normalized to 35 μmol/L in blood plasma. To this effect, ULN can vary based on testing methodology (e.g., enzymatic versus colorimetric, μmol/L versus μg/mL) and from laboratory to laboratory. Two units, μmol/L and μg/dL, can be used for the ammonia data. The conversion formula is μg/dL×0.5872=μmol/L. Ammonia values from different labs can be normalized to 9-35 μmol/L. However, the standard normal reference range to be used for patients 2 months of age to less than 2 years of age is 28-57 μmol/L. Normalization can be done by applying the scale normalization approach using the following formula:

s=x*(U_{S}/U_{X}),

where s is the normalized laboratory value, x is the original laboratory value, U_{X} is the ULN reference range from the original laboratory, and Us is the ULN of the normal reference range for the standard laboratory. For example, if a value of 10 was obtained from a local laboratory with a normal range of 5 to 25, and one wishes to normalize this value to the standard reference range which was established to be 28 to 57, then by applying the above formula, a normalized value of 23 would be obtained, accordingly:

s=10*(57/25)=23

Collection and measurement of a patient's blood plasma ammonia levels are known to those of skill in the art. Notably, fasting blood plasma ammonia levels demonstrate the least variability and offer a practical means for predicting the risk and frequency of an HA crisis. In some embodiments, the patient's blood plasma ammonia levels are assayed after fasting. In some embodiments, a patient's blood plasma ammonia level is assayed using venous blood samples. However, for the purposes of this disclosure, additional, standardized methods of blood plasma ammonia collection and measurement, such as by finger prick, may also be suitable.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Provided is a method of treating a UCD in a patient under 2 years of age comprising
administering glycerol phenylbutyrate to said patient at an initial daily dose which is administered at an initial frequency of administration; and
if after said administration, said patient exhibits a urinary PAGN>9000 μg/mL and a ratio of plasma PAA to plasma PAGN of <2.5, then administering said glycerol phenylbutyrate to said patient at an increased daily dose while maintaining said initial frequency of administration.

Also provided is a method of treating a UCD in a patient under 2 years of age comprising
administering glycerol phenylbutyrate to said patient at an initial daily dose which is administered at an initial frequency of administration; and
if after said administration, said patient exhibits a urinary PAGN>9000 μg/mL and a ratio of plasma PAA to plasma PAGN of >2.5, then administering said glycerol phenylbutyrate to said patient at an increased frequency of administration while maintaining said daily dose.

Also provided is a method of treating a UCD in a patient under 2 years of age comprising
administering glycerol phenylbutyrate to said patient at an initial daily dose which is administered at an initial frequency of administration; and
if after said administration, said patient exhibits a ratio of plasma PAA to plasma PAGN of >2.5 or a level of plasma PAA of >500 μg/mL, then administering said glycerol phenylbutyrate to said patient at a reduced daily dose while maintaining said initial frequency of administration.

Also provided is a method of treating a UCD in a patient under 2 years of age comprising
administering glycerol phenylbutyrate to said patient at an initial daily dose which is administered at an initial frequency of administration; and
if after said administration, said patient exhibits a ratio of plasma PAA to plasma PAGN of >2.5 or a level of plasma PAA of >500 μg/mL, then administering said glycerol phenylbutyrate to said patient at an increased frequency of administration while maintaining said daily dose.

Also provided is a method of treating a UCD in a patient under 2 years of age comprising
administering glycerol phenylbutyrate to said patient at an initial daily dose which is administered at an initial frequency of administration; and
if after said administration said patient exhibits neurological symptoms, normal ammonia levels, and a ratio of plasma PAA to plasma PAGN of <2.5, then said glycerol phenylbutyrate to said patient should continue to be administered at the same dose and frequency and the patient evaluated for other causes of his or her neurological symptoms.

Also provided is a method of treating a UCD in a patient under 2 years of age comprising
administering glycerol phenylbutyrate to said patient at an initial daily dose which is administered at an initial frequency of administration; and
if after said administration, said patient exhibits elevated ammonia levels, a urinary PAGN<9000 μg/mL, a ratio of plasma PAA to plasma PAGN of ≦2.5, and compliance with initial daily dosing is confirmed, then administering said glycerol phenylbutyrate to said patient at an increased dose while maintaining frequency of administration.

Also provided is a method of treating a UCD in a patient under 2 years of age comprising
administering glycerol phenylbutyrate to said patient at an initial daily dose which is administered at an initial frequency of administration; and
if after said administration, said patient exhibits elevated ammonia levels, a urinary PAGN<9000 μg/mL, and a ratio of plasma PAA to plasma PAGN of >2.5, then administering said glycerol phenylbutyrate to said patient at an increased dose frequency while maintaining the said daily dose.

Also provided is a method administering glycerol phenylbutyrate to a patient in need thereof, wherein said patient is between 2 months of age to less than 2 years of age, comprising
administering daily to the patient a therapeutically effective amount of the glycerol phenylbutyrate in three or more equally divided doses.

The methods disclosed herein presume that the patient is effectively receiving all of the prescribed dose. Some parents have difficult delivering a liquid drug to newborns and, therefore, in some embodiments, the compliance and effectiveness of drug delivery by the patients' parent(s), guardian(s), or health care provider(s) can also be assessed.

In some embodiments, neurological symptoms include headache, confusion, vomiting, lethargy, or any combination thereof.

In some embodiments, the UCD is any subtype. In some embodiments, the UCD is not N-acetyl glutamate synthetase deficiency.

In some embodiments, the patient is a newborn, i.e., from birth to one month of age. In some embodiments, the patient is an infant/toddler, i.e., from 1 month to two years of age. In some embodiments, the patients is from two months to two years of age.

In some embodiments, the patient is treated for at least one month. In some embodiments, the patient is treated for at least six months. In some embodiments, the patient is treated for up to 24 months. In some embodiments, the patient is treated for more than 24 months. In some embodiments, the patient is treated for the rest of the patient's life. In some embodiments, the patient treated with the glycerol phenylbutyrate on a relatively continuous daily basis for at least 6 months or longer. In some embodiments, the patient receives a liver transplant and the treatment is terminated.

In some embodiments, the glycerol phenylbutyrate is administered orally. In some embodiments, the glycerol phenylbutyrate is administered via oral syringe. In some embodiments, the glycerol phenylbutyrate is administered via a gastric or nasogastric tube.

In some embodiments, the glycerol phenylbutyrate is administered just prior to breastfeeding or intake of formula or food.

In some embodiments, the patient is not being treated with carglumic acid.

In some embodiments, the patient is in a hyperammonemic crisis and the initial daily dose is 11.2 mL/m²/day. In some embodiments, the patient is not in a hyperammonemic crisis and said initial daily dose is 8.5 mL/m²/day.

In some embodiments, the patient previously had been administered NaPBA or NaBz and said initial daily dose is equal to 0.81 times the total number of grams of NaPBA powder the patient was receiving, or 0.86 times the total number of grams of in NaPBA tablet form the patient was receiving, or 0.5 times the total number of grams of in NaBz the patient was receiving.

In some embodiments, the initial frequency of administration is 3 to 6 times daily.

In some embodiments, if the initial frequency of administration is 3 times a day, then the increased frequency of administration is 4, 5, or 6 times a day. In some embodiments, if the initial frequency of administration is 3 times a day, then the increased frequency of administration is 4 times a day. In some embodiments, if the initial frequency of administration is 3 times a day, then the increased frequency of administration is 5 times a day. In some embodiments, if the initial frequency of administration is 3 times a day, then the increased frequency of administration is 6 times a day.

In some embodiments, if the initial frequency of administration is 4 times a day, then the increased frequency of administration is 5 or 6 times a day. In some embodiments, if the initial frequency of administration is 4 times a day, then the increased frequency of administration is 5 times a day. In some embodiments, if the initial frequency of administration is 4 times a day, then the increased frequency of administration is 6 times a day.

In some embodiments, if the initial frequency of administration is 5 times a day, then the increased frequency of administration is 6 times a day.

In some embodiments, the patient is less than about one month of age.

In some embodiments, the patient is from 1 month to two years of age.

In some embodiments, the patient is from 2 months to 2 years of age.

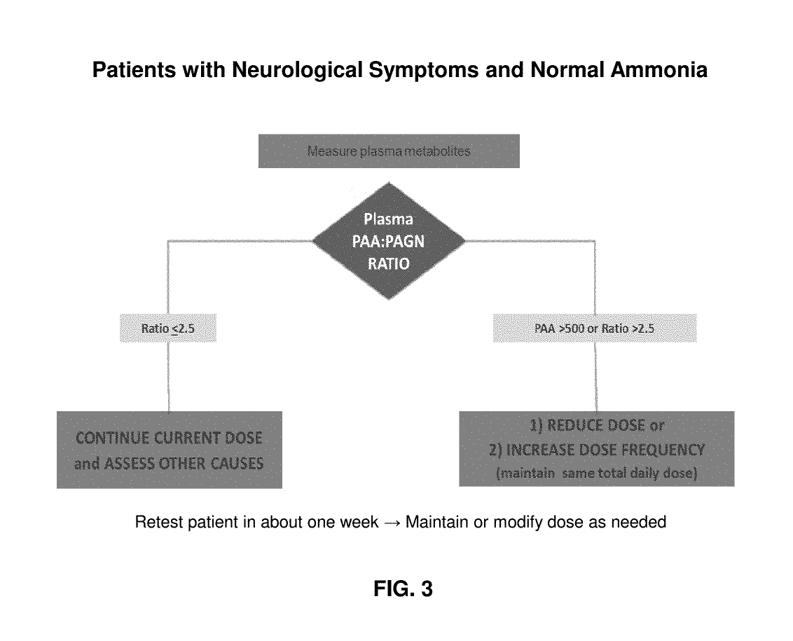

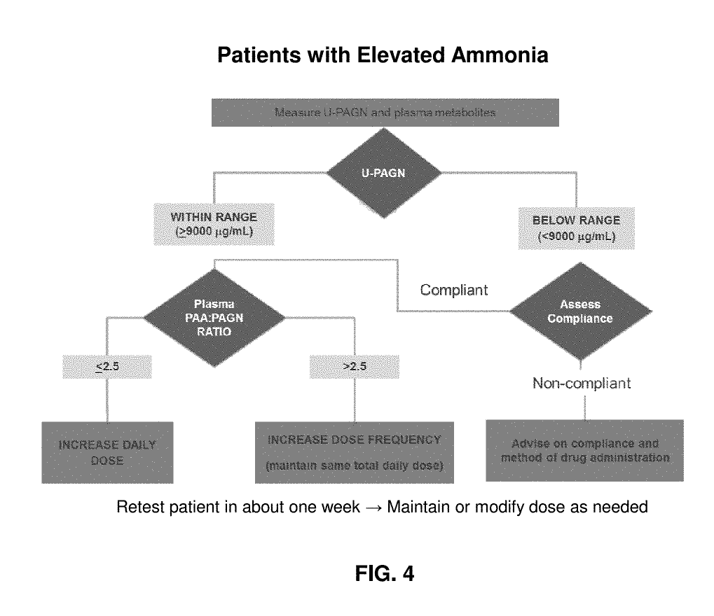

In some embodiments, patient compliance with dosing is checked if U-PAGN is less than 9000 μg/mL and the patient experiences hyperammonemia. See FIGS. 3 and 4.

In some embodiments, the glycerol phenylbutyrate is provided as RAVICTI® product.

In some embodiments, the method further comprises restricting dietary protein, including amino acids and protein formula/supplements. The diet prescribed for each individual depends on developmental needs, age and residual enzyme activity.

In some embodiments, the method further comprises sampling the patient's urine and/or plasma.

In some embodiments, the method further comprises determining the effectiveness of intestinal hydrolysis of the glycerol phenylbutyrate.

In some embodiments, the method further comprises determining the rate at which the patient converts PBA to urinary PAGN.

In some embodiments, the method further comprises determining the efficiency of conjugation of PAA and glutamine.

In some embodiments, the daily dose or frequency of administration is selected and/or adjusted based at least in part on plasma ammonia level, ratio of PAA:PAGN and/or U-PAGN level.

A morning ammonia level less than half the upper limit of normal (the upper limit of normal (ULN) is 35 μmol/L in blood plasma) increases the likelihood that the average daily ammonia will be within normal limits and is associated with a decreased risk and frequency of hyperammonemic crises. In some embodiments, satisfactory ammonia control is defined as: no signs and symptoms of hyperammonemia and blood plasma ammonia values <100 μmol/L. In some embodiments, patient dosing is maintained at an initial dose so long as the patient's blood plasma ammonia level is less than half the ULN (i.e., 35μmol/L). In some embodiments, the ULN is normalized to 35 μmol/L in blood plasma. To this effect, ULN can vary based on testing methodology and from laboratory to laboratory.

In some embodiments, the samples are collected prior to the first dose of the day.

In some embodiments, the duration of ammonia monitoring will vary depending on the patient's age and their presenting conditions, as shown below. Patients less than 2 months of age will in some embodiments have ammonia levels monitored for a total of 72 hours (including 48 hours after the first full dose of glycerol phenylbutyrate) prior to the physician making the judgment that the patient has recovered (no signs and symptoms of hyperammonemia and ammonia <100 μmol/L); patients aged 2 months and older following a hyperammonemic crisis will in some embodiments have ammonia levels monitored for a total of 48 hours (including 24 hours after their first full dose of glycerol phenylbutyrate); and patients aged 2 months and older who are newly diagnosed and stable or are already stable on NaPBA or NaBz will initiate or transition to glycerol phenylbutyrate and have their ammonia levels monitored in some embodiments for 24 hours. A monitoring period of 48-72 hours, depending on the age of the patient, may in some embodiments involve evaluations of ammonia levels at a minimum of every 12 hours.

TABLE I
Determination of Duration for Ammonia Monitoring Period.

| | | Ammonia |
|---|---|---|
| Age of | HA | Monitoring |
| Patient | Crisis | Period |
| <2 months | Yes | 72 hours |
| | No* | 72 hours |
| ≧2 months | Yes | 48 hours |
| | No* | 24 hours |

*Stable Patient: defined as patients not in HA crisis who are diagnosed through newborn screening or patients currently on NaPBA or NaBz.

Population pharmacokinetic (PK) modeling and dosing simulations have demonstrated that body surface area is a determinant of the rate at which patients convert PAA to PAGN for both NaPBA and glycerol phenylbutyrate. Very young patients are more likely than older patients to experience elevated PAA levels (i.e., in the range associated with generally reversible adverse events among cancer patients receiving PAA intravenously (nausea, vomiting, sweating, headache)). Measuring the plasma ratio of PAA to PAGN (the ratio of precursor to product, both in μg/mL) is clinically useful in that it represents an inherent measure of the efficiency with which PAA is converted to PAGN in an individual patient. A PAA:PAGN concentration ratio ≦2.5 indicates efficient conversion of PAA to PAGN and suggests that the dose of NaPBA or GPB phenylbutyrate could be increased, if necessary.

Among all populations and doses studied, a ratio greater than 2.5, when both PAA and PAGN are expressed in μg/mL, is associated with probabilities of PAA levels exceeding 400 μg/mL ranging from approximately 25% to 36%, whereas a ratio less than or equal to 2.5 is associated with an approximately 1% risk of a PAA value >400 μg/mL. Thus, a PAA to PAGN ratio >2.5 in a patient with unexplained neurological symptoms and normal ammonia provides guidance that cautious changes to dose or dosing regimen should be considered while maintaining the same daily dose. Neurological symptoms commonly associated with UCD are known to those of skill in the art and can include somnolence, headaches, lethargy, and confusion. Gastrointestinal symptoms commonly associated with UCD are also known to those of skill in the art and can include vomiting and loss of appetite.

U-PAGN measurements are used to help guide glycerol phenylbutyrate dosing. Each gram of U-PAGN excreted over 24 hours covers waste nitrogen generated from 1.4 grams of dietary protein. Urinary PAGN has been shown to correlate directly and strongly with the dose of PBA administered, either as glycerol phenylbutyrate or NaPBA, and the conversion of PBA to U-PAGN is generally between 60-75%. In young pediatric patients, even where complete urine collection is not possible, use of U-PAGN concentration is useful as a marker of compliance/effective drug delivery. Based on analysis of data obtained from all patients in glycerol phenylbutyrate clinical trials and using the lower 25 percentile as a cutoff, the patient's caretaker should assess compliance and/or effectiveness of drug administration if the U-PAGN is <9000 μg/mL for patients under 2 years of age who exhibit unexplained hyperammonemia during treatment with glycerol phenylbutyrate.

In some embodiments, the patient's desired growth and development and/or body surface area (BSA) is also considered. For example, if the patient's weight/BSA, metabolic needs and/or dietary protein intake have increased, the glycerol phenylbutyrate dose may be increased accordingly; each additional gram of daily protein can be covered by 0.6 mL/day of additional glycerol phenylbutyrate.

In some embodiments, the dosage range, based upon body surface area, in patients naïve to phenylbutyrate (PBA) is 4.5 to 11.2 mL/m²/day (5 to 12.4 g/m²/day). For patients with some residual enzyme activity who are not adequately controlled with protein restriction, in some embodiments, the starting dosage is 4.5 mL/m²/day. In determining the starting dosage of glycerol phenylbutyrate in treatment-naïve patients, consider the patient's residual urea synthetic capacity, dietary protein requirements, and diet adherence. Dietary protein is approximately 16% nitrogen by weight. Given that approximately 47% of dietary nitrogen is excreted as waste and approximately 70% of an administered PBA dose will be converted to urinary phenylacetylglutamine (U-PAGN), an initial estimated glycerol phenylbutyrate dose for a 24-hour period is 0.6 mL glycerol phenylbutyrate per gram of dietary protein ingested per 24-hour period. In some embodiments, the total daily dosage does not exceed 17.5 mL.

Patients 2 months of age to less than 2 years can receive glycerol phenylbutyrate in 3 or more equally divided dosages, each rounded up to the nearest 0.1 mL. When plasma ammonia is elevated, glycerol phenylbutyrate dosage can be increased to reduce the fasting ammonia level to less than half the upper limit of normal (ULN) in patients 6 years and older. In infants and pediatric patients (generally below 6 years of age), where obtaining fasting ammonia is problematic due to frequent feedings, dosage can be adjusted to keep the first ammonia of the morning below the ULN.

In some embodiments, patients who can swallow take glycerol phenylbutyrate orally, even those with nasogastric and/or gastrostomy tubes. For patients who require a volume of less than 1 mL per dose via nasogastric or gastrostomy tube, the delivered dosage may be less than anticipated due to adherence of glycerol phenylbutyrate to the plastic tubing. In some embodiments, these patients are monitored using ammonia levels following initiation of glycerol phenylbutyrate dosing or dosage adjustments.

Because plasma/urine PK results may not be available for real-time dose adjustment decisions, i.e. until after the patient's visit for sampling to be done, the present methods can also be practiced so as to guide dosing by providing a way to evaluate retrospectively dose adjustments already made based on ammonia levels and determine whether further adjustment is appropriate and what it should be, if so. Generally, samples for measurements of PAA and PAGN (plasma) and PAGN (urine) are collected at the time of each dose adjustment (in the retrospective mode; alternatively, samples can be taken and evaluated, and the dose adjustment, if any, implemented after the evaluation results are considered and used to guide dose adjustment, as provided herein).

### EXAMPLES

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1

HPN-100-012, an open-label, fixed sequence, switch-over study of the safety, PK and efficacy of HPN-100 compared to NaPBA in patients with UCDs, with a 12-month safety extension part. The switch-over part of the study and the 12-month safety extension are complete. The switch-over part was conducted at 7 U.S. centers and enrolled 15 pediatric UCD patients between 29 days and <6 year of age, all of whom completed the study. After review of all the safety data by the DSMB, 8 additional subjects were enrolled in the study after the switch-over phase was completed. The primary efficacy endpoint was the 24-hour AUC (area under the curve) for blood ammonia on Day 1 compared with Day 10. Results of this analysis demonstrated that HPN-100 was effective and non-inferior to NaPBA in controlling blood ammonia in pediatric patients with UCDs aged 29 days to <6 years (upper 90% CI of 1.002 and 95% CI of 1.055 based on the original scale, both well below the predefined non-inferiority upper margin of 1.25). The mean ammonia AUC was lower on HPN-100 than on NaPBA (median difference between treatments of −37.84 μmol/L*h); the corresponding p-value was 0.075 using the paired t-test and 0.033 using the nonparametric Wilcoxon rank-sum test.

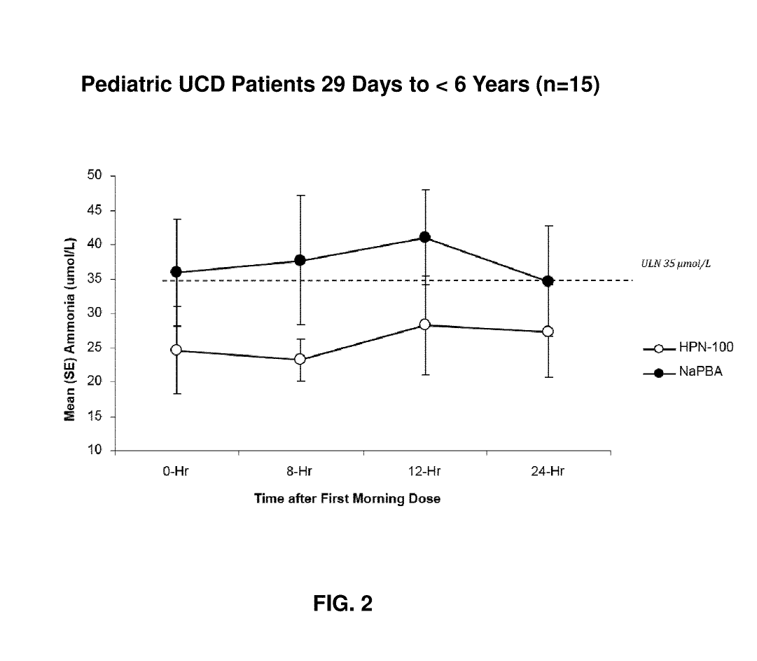

Mean blood ammonia levels following an overnight fast were normal at pre-dose and 24 hours post dose after treatment with NaPBA and HPN-100. See FIG. 2. At any given time point postprandially, blood ammonia levels were directionally lower during treatment with HPN-100 (Day 10) than with NaPBA (Day 1). Mean blood ammonia values were highest for both treatments at the 12-hour time point (28.24 μmol/L on HPN-100 and 41.03 μmol/L on NaPBA) and were lowest for HPN-100 treatment at the 8-h time point (23.20 μmol/L) and 24-hour time point on NaPBA (34.66 μmol/L).

Pooled ammonia analyses from short term studies were also conducted. In the pooled analyses, 24 hour ammonia was significantly lower on HPN-100 vs. NaPBA (mean [SD] AUC₀₋₂₄: 627 [302] vs. 872 [516] μmol/L; p=0.008) with significantly fewer abnormal values (15% on GPB vs. 35% on NaPBA; p=0.02).

The pharmacokinetics of the short term studies can be summarized as follows. Mean exposure levels of HPN-100 metabolites PBA, PAA, and PAGN after doses of HPN-100 and NaPBA with meals (TID) in pediatric (29 days to 17 years of age) UCD patients in HPN-100-005 and HPN-100-012 studies are summarized below. The mean exposure to PBA, the parent metabolite, and mean exposure to PAGN, the terminal metabolite, show no systematic variation across age groups, while PAA exposure tended to decrease with increasing age, a finding consistent with those from population PK modeling which indicate that the rate of clearance/metabolism of PAA varies directly with body size. While systemic metabolite exposure, assessed as peak plasma concentration (Cₘₐₓ) or AUC, is generally similar for HPN-100 and NaPBA, the minimum concentration (Cₘᵢₙ₋ₛₛ) was higher—and/or percent-fluctuation less—for plasma PAGN, a finding consistent with slower gastrointestinal absorption of PBA when administered orally as HPN-100.

TABLE II
Pharmacokinetics of HPN-100 and NaPBA in Pediatric Patients with UCDs.

| | Pediatric UCD Patients (29 days-17 years) | | | |
|---|---|---|---|---|
| | HPN-100-005 (N = 11) | | HPN-100-012 (N = 15) | |
| PK Variable^{a} | HPN-100 | NaPBA | HPN-100 | NaPBA |
| Mean (SD) Dose | 11.04 | 10.94 | 5.16 | 5.27 (2.453) |
| | (3.859) | (3.873) | (2.316) | |
| Plasma PBA | | | | |
| AUC₀₋₂₄ (μg · h/mL) | 631 (44.9) | 236 (105.2) | 255 (54.5) | 483 (146.0) |
| Cₘₐₓ₋ₛₛ (μg/mL) | 95.6 (42.0) | 37.4 (101.6) | 36.8 (59.0) | 60.4 (128.3) |
| Cₘᵢₙ₋ₛₛ (μg/mL) | 1.50 (99.8) | 0.37 (171.3) | 0.750 | 0.993 |
| | | | (192.7) | (227.3) |
| Tₘₐₓ₋ₛₛ (h)^{b} | 12.00 | 12.00 | 8.00 | 8.30 |
| | (3.82, | (8.00, 13.58) | (0.00, | (7.33- |
| | 15.90) | | 12.00) | 12.17) |
| CLss/F (mL/min) | 20545 | 145463 | 22610 | 112093 |
| | (47.2) | (123.4) | (48.6) | (136.4) |
| % Fluctuation | 5690 (56.8) | 1979 (123.7) | 1781 (84.6) | 5102 (75.9) |
| Plasma PAA | | | | |
| AUC₀₋₂₄ (μg · h/mL) | 964 (63.6) | 773 (73.3) | 1096 | 1458 |
| | | | (214.0) | (211.3) |
| Cₘₐₓ₋ₛₛ (μg/mL) | 90.5 (69.1) | 75.1 (64.4) | 84.7 | 98.0 (152.1) |
| | | | (148.3) | |
| Cₘᵢₙ₋ₛₛ (μg/mL) | 2.99 (122.1) | 0.674 | 26.1 | 49.2 (287.2) |
| | | (130.5) | (360.8) | |
| Tₘₐₓ₋ₛₛ (h) | 11.92 | 12.00 | 8.00 | 8.00 |
| | (7.75, | (8.00, 12.02) | (7.42, | (6.50- |
| | 23.83) | | 12.00) | 12.17) |
| CLss/F (mL/min) | 15082 | 34391 | 25538 | 94249 |
| | (101.4) | (162.5) | (116.8) | (243.7) |
| % Fluctuation | 3483 (51.5) | 3931 (85.1) | 2141 | 1289 |
| | | | (145.0) | (102.8) |
| Plasma PAGN | | | | |
| AUC₀₋₂₄ (μg · h/mL) | 1378 (40.2) | 1015 (44.7) | 1131 (71.2) | 946 (75.5) |
| Cₘₐₓ₋ₛₛ (μg/mL) | 105 (33.5) | 74.8 (37.3) | 85.5 (53.7) | 74.4 (61.3) |
| Cₘᵢₙ₋ₛₛ (μg/mL) | 13.1 (64.9) | 4.6 (66.4) | 19.9 | 17.7 (159.6) |
| | | | (173.4) | |
| Tₘₐₓ₋ₛₛ (h) | 12.00 | 12.00 | 7.92 | 8.00 |
| | (7.75, | (7.73, 12.25) | (0.00, | (0.00- |
| | 23.83) | | 12.00) | 12.17) |
| CLss/F (mL/min) | 14237 | 19739 (44.3) | 10830 | 14360 |
| | (36.8) | | (85.6) | (93.0) |
| % Fluctuation | 1001 (84.5) | 1917 (54.8) | 907 (116.7) | 1459 (74.1) |
| U-PAGN | | | | |
| Ae (μg) | 12501037 | 12512426 | NA ^{c} | NA ^{c} |
| | (56.9) | (51.3) | | |
| Ae₀₋₁₂ (μg) | 5611719 | 7098020 | NA ^{c} | NA ^{c} |
| | (74.7) | (47.5) | | |
| Ae₁₂₋₂₄ (μg) | 6889318 | 5414406 | NA ^{c} | NA ^{c} |
| | (50.1) | (71.9) | | |
| Fe % dose | 66.4 (23.9) | 69.0 (23.9) | NA ^{c} | NA ^{c} |
| Fe₀₋₁₂ (%) | 28.9 (42.1) | 39.6 (30.0) | NA ^{c} | NA ^{c} |
| Fe₁₂₋₂₄ (%) | 37.4 (32.3) | 29.3 (52.8) | NA ^{c} | NA ^{c} |

^{a}Unless otherwise noted, data shown are arithmetic mean (coefficient of variation %).
^{b}Median (minimum, maximum).
^{c} Timed urine collections were not feasible among children under 6 participating in protocol HPN-100-012 and therefore recovery of PBA as urinary PAGN could not be calculated. Concentration of PAGN or the PAGN/creatinine ratio was measured/calculated from urine samples, and the results are summarized above.

### Example 2

Uncontrolled, open-label studies were conducted to assess monthly ammonia control and hyperammonemic crisis of RAVICTI® in pediatric patients with UCDs 2 months to less than 2 years of age (Study 4/4E, Study 5, and Study 6). Patients in Study 5 previously participated in Study 4/4E. A total of 17 pediatric patients with UCDs aged 2 months to less than 2 years participated in the studies.

### Uncontrolled, Open-Label Study in Children Under 2 Years of Age (Study 6)

A total of 10 pediatric patients with UCDs aged 2 months to less than 2 years participated in Study 6, of which 7 patients converted from sodium phenylbutyrate to RAVICTI®. The dosage of RAVICTI® was calculated to deliver the same amount of PBA as the sodium phenylbutyrate dosage the patients were taking when they entered the trial. Two patients were treatment naïve and received RAVICTI® dosage of 7.5 mL/m²/day and 9.4 mL/m²/day, respectively. One additional patient was gradually discontinued from intravenous sodium benzoate and sodium phenylacetate while RAVICTI® was initiated. The dosage of RAVICTI® after transition was 8.5 mL/m²/day.

In Study 6, there were 9, 7 and 3 pediatric patients who completed 1, 3 and 6 months, respectively (mean and median exposure of 4 and 5 months, respectively).

Patients received a mean RAVICTI® dose of 8 mL/m²/day (8.8 g/m²/day), with doses ranging from 4.8 to 11.5 mL/m²/day (5.3 to 12.6 g/m²/day). Patients were dosed three times a day (n=6), four times a day (n=2), or five or more times a day (n=2).

The primary efficacy endpoint was successful transition to RAVICTI® within a period of 4 days followed by 3 days of observation for a total of 7 days, where successful transition was defined as no signs and symptoms of hyperammonemia and a venous ammonia value less than 100 micromol/L. Venous ammonia levels were monitored for up to 4 days during transition and on day 7. Nine patients successfully transitioned as defined by the primary endpoint. One additional patient developed hyperammonemia on day 3 of dosing and experienced surgical complications (bowel perforation and peritonitis) following jejunal tube placement on day 4. This patient developed hyperammonemic crisis on day 6, and subsequently died of sepsis from peritonitis unrelated to drug. Although two patients had day 7 ammonia values of 150 micromol/L and 111 micromol/L respectively, neither had associated signs and symptoms of hyperammonemia.

During the extension phase, venous ammonia levels were monitored monthly. Ammonia values across different laboratories were normalized (transformed) to a common normal pediatric range of 28 to 57 micromol/L for comparability. The mean normalized venous ammonia values in pediatric patients at month 1, 2, 3, 4, 5 and 6 were 67, 53, 78, 99, 56 and 61 micromol/L during treatment with RAVICTI®, respectively. Three patients reported a total of 7 hyperammonemic crises defined as having signs and symptoms consistent with hyperammonemia (such as frequent vomiting, nausea, headache, lethargy, irritability, combativeness, and/or somnolence) associated with high venous ammonia levels and requiring medical intervention. Hyperammonemic crises were precipitated by vomiting, upper respiratory tract infection, gastroenteritis, decreased caloric intake or had no identified precipitating event (3 events). There were three additional patients who had one venous ammonia level that exceeded 100 micromol/L which was not associated with a hyperammonemic crisis.

### Uncontrolled, Open-Label Studies in Children Under 2 Years of Age (Studies 4/4E, 5)

A total of 7 patients with UCDs aged 2 months to less than 2 years participated in Studies 4/4E and 5. In these studies, there were 7, 6, 6, 6 and 3 pediatric patients who completed 1, 6, 9, 12 and 18 months, respectively (mean and median exposure of 15 and 17 months, respectively). Patients were converted from sodium phenylbutyrate to RAVICTI®. The dosage of RAVICTI® was calculated to deliver the same amount of PBA as the sodium phenylbutyrate dosage the patients were taking when they entered the study.

Patients received a mean RAVICTI® dose of 7.5 mL/m²/day (8.2 g/m²/day), with doses ranging from 3.3 to 12.3 mL/m²/day (3.7 to 13.5 g/m²/day). Patients were dosed three times a day (n=3) or four times a day (n=4).

Venous ammonia levels were monitored on days 1, 3 and 10 in Study 4 and at week 1 in Study 4E. Two patients had day 1 ammonia values of 122 micromol/L and 111 micromol/L respectively, neither had associated signs and symptoms of hyperammonemia. At day 10/week 1, six of the 7 patients had venous ammonia levels less than 100 micromol/L the remaining patient had a day 10 ammonia value of 168 micromol/L and was asymptomatic.

During the extension period, venous ammonia levels were monitored monthly. Ammonia values across different laboratories were normalized (transformed) to a common normal pediatric range of 28 to 57 micromol/L for comparability. The mean venous ammonia values in pediatric patients at month 1, 3, 6, 9 and 12 were 58, 49, 34, 65, and 31 micromol/L during treatment with RAVICTI®, respectively.

Three patients reported a total of 3 hyperammonemic crises, as defined in Study 6. Hyperammonemic crises were precipitated by gastroenteritis, vomiting, infection or no precipitating event (one patient). There were 4 patients who had one venous ammonia level that exceeded 100 micromol/L which was not associated with a hyperammonemic crisis.

What is claimed is:

1. A method of administering glycerol phenylbutyrate to a patient in need thereof, wherein said patient is between 2 months of age to less than 2 years of age, comprising
administering daily to the patient a therapeutically effective amount of the glycerol phenylbutyrate in three or more equally divided doses.

2. The method of claim 1, further comprising restricting the patient's dietary protein.

3. The method of claim 1, further comprising monitoring the patient's plasma ammonia levels to determine the need for dosage titration of the glycerol phenylbutyrate.

4. The method of claim 3, further comprising adjusting the glycerol phenylbutyrate dosage to keep the first ammonia of the morning below the upper limit of normal.

5. The method of claim 1, wherein the glycerol phenylbutyrate is administered orally.

6. The method of claim 1, wherein the glycerol phenylbutyrate is administered using a nasogastric and/or gastrostomy tube.

7. The method of claim 6, wherein the volume of glycerol phenylbutyrate administered is less than 1 mL per dose and the method further comprises monitoring the patient's ammonia level.

8. The method of claim 1, wherein the patient previously was administered phenylbutyrate.

9. The method of claim 1, wherein the patient previously had not been administered phenylbutyrate.

10. The method of claim 1, wherein the therapeutically effective amount of the glycerol phenylbutyrate is 4.5 to 11.2 mL/m²/day (5 to 12.4 g/m²/day).

11. The method of claim 1, wherein the therapeutically effective amount of the glycerol phenylbutyrate is 4.8 to 11.5 mL/m²/day (5.3 to 12.6 g/m²/day).

12. The method of claim 1, wherein the therapeutically effective amount of the glycerol phenylbutyrate is 3.3 to 12.3 mL/m²/day (3.7 to 13.5 g/m²/day).

13. The method of claim 1, wherein the therapeutically effective amount of the glycerol phenylbutyrate is 4.5 mL/m²/day (5 g/m²/day).

14. The method of claim 1, wherein the therapeutically effective amount of the glycerol phenylbutyrate is 8 mL/m²/day (8.8 g/m²/day).

15. The method of claim 1, wherein the therapeutically effective amount of the glycerol phenylbutyrate is 7.5 mL/m²/day (8.2 g/m²/day).

16. The method of claim 1, further comprising obtaining measurements of plasma phenylacetate (PAA) concentrations and the ratio of plasma PAA to phenylacetylglutamine (PAGN).

17. The method of claim 1, further comprising obtaining measurements of urinary phenylacetylglutamine (U-PAGN).

18. The method of claim 17, wherein if the U-PAGN excretion is insufficient to cover daily dietary protein intake and/or the fasting ammonia is greater than half the upper limit of normal, the method further comprises increasing the glycerol phenylbutyrate dosage.

19. The method of claim 1, wherein the glycerol phenylbutyrate is administered just prior to breastfeeding or intake of formula or food.

20. The method of claim 1, wherein the glycerol phenylbutyrate is administered three to six times daily.
